SEQUENCE LISTING (8 sequences)

SEQ ID NO: 1 - DNA, 30 bp, Homo sapiens
gcctccaacggaaaccagtctcagtgtcca

SEQ ID NO: 2 - DNA, 29 bp, Homo sapiens
ccgtccgagggttgtatccgctgctttgt

SEQ ID NO: 3 - DNA, 20 bp, Artificial
  Synthetic DNA
tgtaaaacgacggccagtcc

SEQ ID NO: 4 - DNA, 18 bp, Artificial
  Synthetic DNA
actggccgtcgttttaca

SEQ ID NO: 5 - DNA, 20 bp, Artificial
  Synthetic DNA
caggaaacagctatgaccgt

SEQ ID NO: 6 - DNA, 18 bp, Artificial
  Synthetic DNA
ggtcatagctgtttcctg

SEQ ID NO: 7 - DNA, 18 bp, Artificial
  Synthetic DNA
tgtaaaacgacggccagt

SEQ ID NO: 8 - DNA, 18 bp, Artificial
  Synthetic DNA
caggaaacagctatgacc

(19) United States
(12) Patent Application Publication
(10) Pub. No.: US 2008/0131937 A1
(43) Pub. Date: Jun. 5, 2008

(54) Conversion of Target Specific Amplification to Universal Sequencing

(75) Inventor: Benjamin G. Schroeder, San Mateo, CA (US)

Correspondence Address: MILA KASAN, PATENT DEPT.;APPLIED BIOSYSTEMS, 850 LINCOLN CENTRE DRIVE, FOSTER CITY, CA, 94404, US

(73) Assignee: APPLERA CORPORATION, Foster City, CA (US)

(21) Appl. No.: 11/764,115

(22) Filed: Jun. 15, 2007

Related U.S. Application Data
(60) Provisional application No. 60/805,560, filed on Jun. 22, 2006.

(51) Int. Cl. C12P 19/34; C12N 9/12

(52) U.S. Cl. 435/91.2; 435/194

(57) ABSTRACT

The present teachings provide methods, compositions, and kits for amplifying nucleic acids. The amplified nucleic acids can then be sequenced. The sequencing reaction for such amplified nucleic acids can provide additional sequence information, and less redundancy, as compared to conventional approaches. In some embodiments, a target polynucleotide is amplified with a primer to form an amplicon comprising a type IIs restriction enzyme recognition site. Following digestion with a type IIs restriction enzyme, and ligation of an adapter, a sequencing primer can be hybridized to the adapter, and a sequencing reaction performed. The sequencing reaction results in the omission of unwanted sequence information that was present in the amplification primer.

### CROSS-REFERENCE TO RELATED APPLICATIONS

This application claims a priority benefit under 35 U.S.C. § 119(e) from U.S. Application No. 60/805,560, filed Jun. 22, 2006, the contents of which are incorporated herein by reference.

### FIELD

The present teachings generally relate to methods for amplifying nucleic acid that provide improvements in nucleic acid sequencing.

### INTRODUCTION

Many next generation DNA sequencing technologies promise very high bases per day throughput, but employ relatively short read-lengths (see for example Metzker, Genome Research (2005) 15:1767-1776). This increased throughput brings the promise of whole genome sequencing. Nonetheless, targeted re-sequencing still offers advantages in cost, time, and sequence depth over these whole genome approaches. One aspect of re-sequencing technologies that employ short read-length is that most are currently configured to use a single set of universal PCR and sequencing primers. If one uses a universally tailed PCR primer approach on a sequencing technology that offers, for example, 25 base-pair reads, the first 18-22 bases can be the gene-specific PCR, a sequence the experimentalist already knows. As a result, only 3-7 bases of actual novel sequence information would be collected in such a 25 base-pair run.

### SUMMARY

The present teachings provide a method of performing a sequencing reaction, wherein the first sequenced base is target polynucleotide information, said method comprising; performing a primer-mediated amplification reaction to form an amplicon, wherein the primer comprises a target-specific portion and cleavage-related moiety; digesting the PCR amplicon with a cleavage agent directed to the cleavage-related moiety to form a digested amplicon with a cleaved end; ligating an adapter to the cleaved end of the digested amplicon to form an adapter-ligated amplicon; and, sequencing the adapter-ligated amplicon, wherein the first sequenced base is target polynucleotide information. Compositions and kits are also provided.

### DESCRIPTION OF EXEMPLARY EMBODIMENTS

It is to be understood that both the foregoing general description and the following detailed description are exemplary and explanatory only and are not intended to limit the scope of the current teachings. In this application, the use of the singular includes the plural unless specifically stated otherwise. For example, “a primer” means that more than one primer can, but need not, be present; for example but without limitation, one or more copies of a particular first primer species, as well as one or more versions of a particular primer type, for example but not limited to, a multiplicity of different forward primers. Also, the use of “comprise”, “comprises”, “comprising”, “contain”, “contains”, “containing”, “include”, “includes”, and “including” are not intended to be limiting.

The section headings used herein are for organizational purposes only and are not to be construed as limiting the described subject matter in any way. All literature and similar materials cited in this application, including but not limited to, patents, patent applications, articles, books, treatises, and internet web pages are expressly incorporated by reference in their entirety for any purpose. In the event that one or more of the incorporated literature and similar materials contradicts this application, including but not limited to defined terms, term usage, described techniques, or the like, this application controls.

### Exemplary Embodiments

The present teachings provide for incorporating a type IIs restriction site, other functionally analogous regions (referred to generally as “cleavage-related moieties”) into the 5′ end of PCR primers. Type IIs enzymes are a special type of restriction enzyme that cuts a defined number of bases away from its recognition site. Several type IIs enzymes are known, which comprise different recognition sites, as well as different lengths between the recognition site and the cut site. For example, MmeI recognizes TCCRAC and cuts 20 and 18 bases 3′ of this sequence, leaving a two base 3′ overhang. As another example, EcoP15I, which recognizes CAGCAG and cuts 25 and 27 bases 3′ of the recognition site, leaving a two base 5′ overhang.

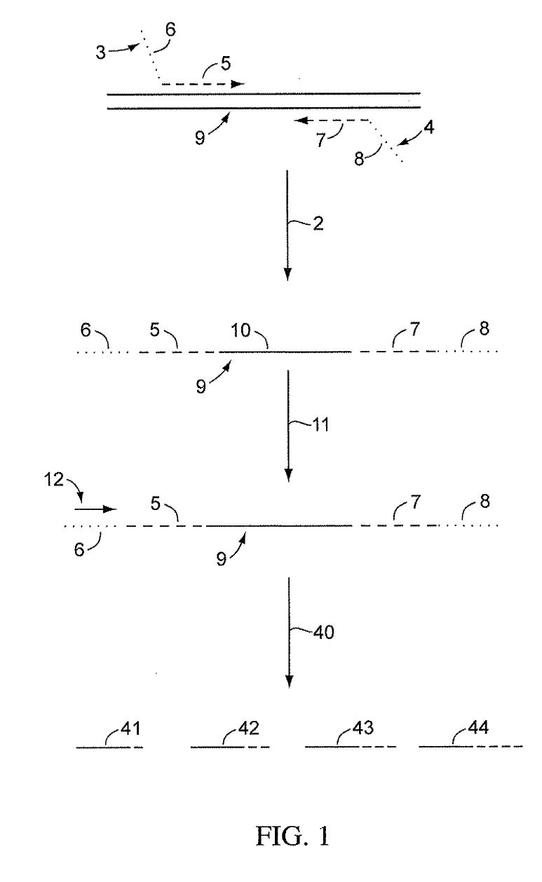

For illustration, a traditional approach to sequencing a target polynucleotide is depicted in FIG. 1. Here, a double-stranded target polynucleotide (1) is amplified in a PCR (2) with a forward primer (3) and a reverse primer (4). The forward primer (3) comprises a target-specific portion (dashed, 5) and a 5′ tail (dotted, 6). The reverse primer (4) comprises a target-specific portion (dashed, 7) and a 5′ tail (dotted, 8). One strand of the resulting PCR amplicon is depicted (9), which comprises the 5′ tail of the forward primer (dotted, 6), the target-specific portion of the forward primer (dashed, 7), the amplified region of the target polynucleotide (10), the target-specific portion of the reverse primer (dashed, 7) and the 5′ tail of the reverse primer (dotted, 8). A sequencing primer (12) can be hybridized (11) to one strand of the PCR amplicon (9), in particular the region of the PCR amplicon corresponding to the 5′ tail of the forward primer (doffed, 6). A sequencing reaction (40) can then be performed, for example, Sanger di-deoxy termination. A collection of Sanger truncation products are shown as 41, 42, 43, and 44. Of note, when using such an approach, the initial sequence information resulting from the Sanger reaction will comprise the determination of nucleic acid sequence already known. Specifically, the first bases of sequencing information will comprise the target-specific portion of the forward primer (dashed, 5) that was part of the forward primer. Products 41, 42, 43, and 44 are simply telling the experimentalist what the sequence was in the target specific portion of the forward primer (5). Thus, the experimentalist is collecting data that comprises no novel sequence information, which is inefficient.

The present teachings provide an improved approach for producing a PCR amplicon that increases the efficiency of collecting novel sequence information. In some embodiments, the amplicon produced by the present teachings can be sequenced, wherein the resulting sequencing reaction reduces the amount of already-known sequence information, and more readily ascertains novel sequence information. The sequencing reactions performed in the context of the present teachings provide for both the collection of novel sequence information that was not present in the at least one primer, as well as the absence of sequence information corresponding to sequence that was present in the at least one primer. By performing a sequencing reaction in which some, or all, of the sequence contained in the PCR primer is omitted, the experimentalist is collecting novel data, and not wasting resources re-determining the already known sequence of the PCR primers.

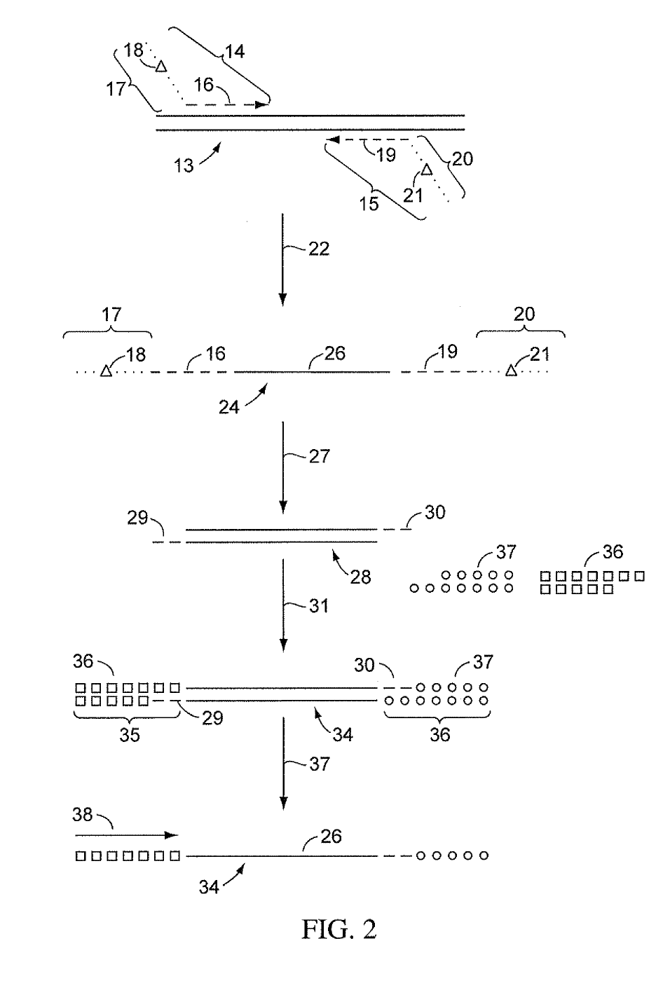

FIG. 2 depicts one approach according to the present teachings whereby a sequencing reaction results in the omission of unwanted sequence information present in the PCR primer. Here, a double-stranded target polynucleotide (13) is amplified in a PCR (22) with a forward primer (14) and a reverse primer (15). The forward primer (14) comprises a target-specific portion (dashed, 16) and a 5′ tail (dotted, 17). A type IIs restriction enzyme recognition site for Mme I (triangle, 18) can be encoded in the 5′ tail of the forward primer (17). The reverse primer (15) comprises a target-specific portion (dashed, 19) and a 5′ tail (dotted, 20). A type IIs restriction enzyme recognition site for Mme 1 (triangle, 21) can be encoded in the 5′ tail of the reverse primer (20). The resulting PCR amplicon comprises two strands, one of which is shown (24). This strand (24) comprises sequence corresponding to the 5′ tail of the forward primer (dotted, 17), the type IIs restriction enzyme recognition site of the 5′ tail of the forward primer (triangle, 18), the target-specific portion of the forward primer (dashed, 16), the amplified region of the target polynucleotide (26), the target-specific portion of the reverse primer (dashed, 19), the 5′ tail of the reverse primer (dotted, 20), and the type IIs restriction enzyme recognition site of the 5′ tail of the reverse primer (triangle, 21).

Treatment with a type IIs restriction enzyme (27) can then be performed to digest the PCR amplicon, thus forming a digested amplicon (28) with a first end (29) and a second end (30). Ligation (31) of a first adapter (36) to the first end of the digested amplicon (29), and ligation of a second adapter (37) to the second end of the digested amplicon (30) can then be performed to form an adapter-ligated amplicon (34) with a first end (35) and a second end (36).

A sequencing reaction (37) can be performed on one strand of the adapter-ligated amplicon (34), wherein a sequencing primer (38) is hybridized, and a sequencing reaction performed such as Sanger di-deoxy termination. Of note, when using the approach depicted here in FIG. 2, the initial sequence information resulting from the Sanger reaction can comprise the determination of novel nucleic acid sequence information. Specifically, the first bases of sequencing information will comprise the amplified region of the target polynucleotide (26). Thus, the experimentalist is collecting new data, and not simply re-determining the already-known sequence of the target-specific portion of the forward primer (16). Such an approach can be more efficient than the approach depicted in FIG. 1. The approach in FIG. 2 provides for a sequencing reaction that can omit the collection of unwanted sequence information present in the PCR primers. Thus, as used herein the term “target polynucleotide information” refers to sequence information that was not already known by virtue of the PCR primer sequence, and is thus sequence information that is downstream of the primer binding sites.

Thus, in some embodiments the present teachings provide a method of performing a re-sequencing reaction comprising; performing a polymerase chain reaction (PCR) with a forward primer and a reverse primer to form a PCR amplicon, wherein the forward primer comprises a target-specific portion and a 5′ tail, wherein the 5′ tail of the forward primer comprises a type IIs restriction enzyme recognition site, and, wherein the reverse primer comprises a target-specific portion and a 5′ tail, wherein the 5′ tail of the reverse primer comprises a type IIs restriction enzyme recognition site; digesting the PCR amplicon with a type IIs restriction enzyme to form a digested amplicon with a first end and a second end; ligating an adapter to the first end of the digested amplicon and ligating an adapter to the second end of the digested amplicon to form an adapter-ligated amplicon with a first end and a second end; and, sequencing the adapter-ligated amplicon.

In some embodiments, the sequencing comprises hybridizing a primer to the first end of the adapter-ligated amplicon; and, performing a chain termination reaction. In some embodiments, the sequencing comprises a cycled ligation. In some embodiments, the adapter ligated to the first end of the digested amplicon is different from the adapter ligated to the second end of the digested amplicon. In some embodiments, the type IIs restriction enzyme is Mme I or EcoP15I.

In some embodiments, the present teachings provide a method of forming an adapter-ligated amplicon comprising a first end that differs in sequence from a second end, said method comprising; amplifying a short nucleic acid in a polymerase chain reaction (PCR) comprising a forward primer and a reverse primer to form a PCR amplicon, wherein the forward primer comprises a target-specific portion and a 5′ tail, wherein the 5′ tail of the forward primer comprises a type IIs restriction enzyme recognition site, and, wherein the reverse primer comprises a target-specific portion and a 5′ tail, wherein the 5′ tail of the reverse primer comprises a type IIs restriction enzyme recognition site; digesting the PCR amplicon with a type IIs restriction enzyme to form a

digested amplicon with a first end and a second end; and, ligating a first adapter to the first end of the digested amplicon and ligating a second adapter to the second end of the digested amplicon to form an adapter-ligated amplicon with a first end that differs in sequence from a second end.

In some embodiments, the PCR amplicon is 18-100 nucleotides in length. In some embodiments, the type IIs restriction enzyme is Mme I or EcoP15I.

In some embodiments, the present teachings provide a method of increasing the percent of novel sequence information in a sequencing reaction of a digested PCR amplicon less than 100 bases in length comprising; performing a polymerase chain reaction (PCR) with a forward primer and a reverse primer to form a PCR amplicon, wherein the forward primer comprises a target-specific portion and a 5′ tail, wherein the 5′ tail of the forward primer comprises a type IIs restriction enzyme recognition site, and, wherein the reverse primer comprises a target-specific portion and a 5′ tail, wherein the 5′ tail of the reverse primer comprises a type IIs restriction enzyme recognition site; digesting the PCR amplicon with a type IIs restriction enzyme to form a digested PCR amplicon with a first end and a second end, wherein the digested PCR amplicon is less than a hundred bases in length; ligating an adapter to the first end of the digested PCR amplicon and ligating an adapter to the second end of the digested PCR amplicon to form an adapter-ligated PCR amplicon with a first end and a second end; sequencing the adapter-ligated amplicon; and, increasing the percent of novel sequence information in a sequencing reaction of the PCR amplicon as compared to sequencing a PCR amplicon that was amplified in a PCR not comprising primers with a type IIs restriction enzyme site.

In some embodiments, the increasing the percent of novel sequence information in a sequencing reaction of the PCR amplicon as compared to sequencing a PCR amplicon that was amplified in a PCR not comprising primers with a type IIs restriction enzyme site comprises greater than ten percent additional sequence information. For example, a target sequence of fifty nucleotides can be defined in reference to the 3′ most nucleotides of the corresponding primers. For such a given fifty nucleotide target sequence, when fifty novel nucleotides are sequenced (e.g. a FIG. 2—like approach), rather than forty-five novel nucleotides and five known nucleotides (a FIG. 1—like approach), it can be said that ten percent additional sequence information is obtained. In some embodiments greater than two percent additional sequence information is obtained. In some embodiments greater than two percent, three percent, four percent, five percent, six percent, seven percent, eight percent, nine percent, ten percent, eleven, twelve, thirteen, fourteen, fifteen, sixteen, seventeen, eighteen, nineteen, twenty, twenty-one, twenty-two, twenty-three, twenty-four, twenty-five, thirty, thirty-five, forty, forty-five, fifty, fifty-five, sixty, seventy, eighty percent, or even greater, additional sequence information is obtained.

In some embodiments, the present teachings provide a method of sequencing a target polynucleotide comprising; amplifying the target polynucleotide with at least one primer to form an amplicon comprising a type IIs restriction enzyme recognition site on at least one end; digesting the PCR amplicon with a type IIs restriction enzyme to form a digested amplicon with at least one ligatable end; ligating an adapter to the ligatable end of the digested amplicon to form an adapter-ligated amplicon with a known end; hybridizing a sequencing primer to the known end; and, sequencing the adapter-ligated amplicon, wherein the first sequenced base is target polynucleotide information.

In some embodiments, the present teachings provide a method of performing a sequencing reaction comprising; performing a polymerase chain reaction (PCR) with a forward primer and a reverse primer to form a PCR amplicon, wherein the forward primer comprises a target-specific portion and a 5′ tail, wherein the 5′ tail of the forward primer comprises an exonuclease-resistant moiety, and, wherein the reverse primer comprises a target-specific portion and a 5′ tail, wherein the 5′ tail of the reverse primer comprises an exonuclease-resistant moiety; digesting the PCR amplicon with an exonuclease to form a digested amplicon with a first end and a second end; ligating an adapter to the first end of the digested amplicon and to the second end of the digested amplicon to form an adapter-ligated amplicon with a first end and a second end; and, sequencing the adapter-ligated amplicon.

In some embodiments, the sequencing comprises hybridizing a primer to the first end of the adapter-ligated amplicon; and, performing a chain termination reaction. In some embodiments, the sequencing comprises a cycled ligation. In some embodiments, the adapter ligated to the first end of the digested amplicon is different from the adapter ligated to the second end of the digested amplicon. In some embodiments, the exonuclease is lambda exonuclease.

In some embodiments, the present teachings provide a method of performing a sequencing reaction, wherein the first sequenced base is target polynucleotide information, said method comprising; performing a primer-mediated amplification reaction to form an amplicon, wherein the primer comprises a target-specific portion and cleavage-related moiety; digesting the amplicon with a cleavage agent directed to the cleavage-related moiety to form a digested amplicon with a cleaved end; ligating an adapter to the cleaved end of the digested amplicon to form an adapter-ligated amplicon; and, sequencing the adapter-ligated amplicon, wherein the first sequenced base is target polynucleotide information. In some embodiments, the sequencing comprises hybridizing a primer to the first end of the adapter-ligated amplicon; and, performing a chain termination reaction. In some embodiments, the sequencing comprises a cycled ligation. In some embodiments, the cleavage-related moiety is a type IIs restriction enzyme recognition site. In some embodiments, the type IIs restriction enzyme is Mme I or EcoP151. In some embodiments, the cleavage-related moiety is an exonuclease-resistant moiety. In some embodiments, the exonuclease-resistant moiety is polyethylene glycol (PEG).

In still other embodiments, the present teachings provide a method of sequencing a target polynucleotide comprising; amplifying the target polynucleotide in a polymerase chain reaction (PCR) to form a PCR amplicon comprising a first strand and a second strand, wherein the first strand comprises a primer bearing an exonuclease resistant moiety, and wherein the second strand is complementary to the first strand; digesting the first strand with an exonuclease to produce a single-stranded region in the second strand, wherein the exonuclease resistant moiety in the primer prevents complete digestion of the first strand, digesting the single-stranded region in the second strand with a single-stranded exonuclease, to form a blunt end; ligating an adapter to the blunt end to form an adapter-ligated amplicon, wherein the adapter comprises a sequencing primer portion; hybridizing a sequencing primer to the sequencing primer portion of the adapter-ligated amplicon; and, sequencing the adapter-ligated amplicon, wherein the first sequenced base is target polynucleotide information.

Any of a variety of type IIs restriction enzymes can be employed according to the present teachings. Illustrative type IIs restriction enzymes, along with their recognition and cut site can be found, for example, in the New England Biolabs product catalog, where such enzymes are commercially available. For a review of type IIs restriction enzymes and further description generally see Aggarwal et al., Curr Opin Struct Biol. 1998 February; 8(1):19-25, U.S. Pat. No. 6,395,523, and U.S. Pat. No. 6,413,758.

While the present teachings are described in the context of a PCR-based amplification, it will be appreciated that any of a variety of amplification reactions can be employed. Such reactions, for example rolling circle amplification (RCA), can comprise primers that are incorporated into amplification products, wherein the primers are designed to contain type IIs restriction site.

Upon the formation of an adapter-ligated amplicon, any of a variety of downstream reactions can be performed. For example, the adapter-ligated amplicon can be sequenced. Sequencing of the adapter-ligated amplicon can be performed using any of a variety of techniques available to the contemporary molecular biologist. For example, a Sanger reaction can be employed where chain termination is achieved by the incorporation of a dideoxy nucleotide (Sanger et al., (1977) PNAS 74:5463-5467). Illustrative examples of Sanger sequencing are well known to one of ordinary skill in the art of molecular, and are further described for example in Sambrook et al., Molecular Cloning, 3^{rd} Edition. In some embodiments, ligation-based sequencing can be employed, as described for example in U.S. Pat. No. 5,403,708 and U.S. Pat. No. 6,306,597. Thus, as used herein, the employment of such ligation-based sequencing uses “sequencing ligation probes.” In other embodiments, a sequencing reaction can be employed in which a reversible terminator is employed, as discussed for example in U.S. Pat. No. 6,664,079. Thus, as used herein, the term “terminator” refers both to conventional di-deoxy (non-reversible) terminators, as well as reversible terminators, including reversible terminators with labels on the base, as well as reversible terminators in which the label is on the sugar. Such approaches are generally referred to herein as “chain termination reactions”. Illustrative reversible terminator teachings include U.S. Pat. No. 6,232,465, U.S. Pat. No. 5,763,594, and U.S. Pat. No. 5,302,509.

The present teachings further contemplate embodiments that use cleavage-related moieties that are not type IIs restriction enzyme recognition sites, but are functionally analogous to type IIs restriction enzyme recognition sites. In some embodiments, the cleavage-related moiety is chemical modification that results in resistance to cleavage by an exonuclease. For example, the inclusion of a phosphorothioate moiety into the phosphate backbone of DNA can render the sequence downstream from (3′ of) the phosphorothioate resistant to exonuclease digestion. Thus, as used herein the term “cleavage-related moiety” refers to both a type IIs recognition site, as well as moieties which provide for the selective removal of some nucleotides of an amplicon, such as an exonuclease-resistant moiety like poly-ethylene glycol (PEG). For example, in some embodiments at least one of the primers in the amplification reaction can comprise a cleavage-related moiety, where following amplification the amplicon can be treated with an exonuclease. This cleavage-related moiety can be in the target-specific portion of the primer or, if the primer has a 5′ tail, can be in the 5′ tail of the primer. Following amplification, primer sequence incorporated into the end of the amplicon, upstream from the phosphorothioate, can be digested by treatment with an exonuclease, whereas amplicon sequence downstream from the cleavage-related moiety remains intact. The other strand of the amplicon, which was rendered single stranded, can then be treated with a single-stranded specific exonuclease, such that the overhanging single-stranded region left by the first exonuclease treatment is removed by the single-stranded specific exonuclease. A blunt end then results. The rest of the amplicon remains in tact. Following these procedures, an adapter can be ligated onto the end of the amplicon that was manipulated in the preceding procedures, thus allowing for the generation of a priming site for a sequencing reaction. Various methods of employing exonucleases to selectively degrade nucleic acids that are applicable to the present teachings can be found further described, for example, in U.S. Pat. No. 6,797,470, and U.S. Pat. No. 7,208,278. In some embodiments, an RNA moiety can be included, and digestion can proceed for example by treating with base, heat, or both.

In some embodiments, a target sequence can be amplified in a PCR that comprises two primers, only one which comprises a cleavage-related moiety. The amplicon resulting from the PCR can be treated with a cleavage agent, such that the target-specific portion of the amplicon end that was derived from the primer containing the cleavage-related moiety is removed. An adapter can be ligated to this resulting end, wherein the adapter comprises a priming site for a sequencing reaction.

In some embodiments, both primers in a PCR comprise cleavage-related moieties, but each primer comprises a different cleavage-related moiety from the other primer. For example, in some embodiments, each primer can comprise a unique type IIs recognition site, such as a forward primer comprising a mmeI site in its tail and a reverse primer comprising an EcoP151 site in its tail. In some embodiments, one primer can comprise a type IIs cleavage-related moiety and the other primer can comprise a chemical modification conferring exonuclease resistance.

In some embodiments, the relative placement of the cleavage-related moiety and the target specific portion of the primer is such that the experimentalist knows what the overhang resulting from cleavage will be, and thus a single adapter, or a single pair of adapters, can be employed to ligate to the ends of a plurality of different digested amplicons. For example, the experimentalist can slide the placement of the target-specific portion of a primer along a sequence of interest in such fashion as to allow for a particular overlap sequence to arise following cleavage of the amplicon. As another example, the experimentalist can vary the number of bases between the cleavage-related moiety and the target-specific portion of the primer in such fashion as to allow for a particular overlap sequence to arise following cleavage of the amplicon. In some embodiments, the experimentalist can employ a degenerate collection of adapters that include all possible sequence of sticky ends, such that a variety of amplicons with different digested ends can be ligated to one member a library of degenerate adapters. The library of degenerate adapters can all comprise the same sequencing primer portion, but vary in their sticky end.

In some embodiments, a multiplexed PCR can be performed where the cleavage-related moieties, (for example, type IIs sites) are zip-coded to a particular target-sequence, and the resulting digested amplicons thus have unique sticky ends depending on the nature of the cleavage-related moiety. The design of the target-specific portion of the primer relative to the type II site in the tail of that primer will typically be such that the eventual cleavage of the amplicon occurs at a site just upstream from the 3′ end of the target specific portion of the primer. Since the resulting sticky end will be known in advance by the experimentalist, a pool of adapters that have sticky ends and sequencing primer binding sites zip-coded to a particular digested amplicon can then be employed. The sequencing primer employed in the eventual sequencing reaction can thus be zip-coded to a particular target sequence.

In some embodiments, a multiplexed PCR can be performed comprising a plurality of target sequence-specific primers. The plurality of target sequence-specific primer pairs can all comprise the same tail, for example the tail of each forward primer can comprise a Mme I site and the tail of each reverse primer can comprise an EcoP15I site. Following digestion with the appropriate cleavage agent, one can ligate universal adapters onto the ends of all the amplicons, for example a first adapter comprising a universal forward priming site directed to one end of an amplicon and a second adapter comprising a universal reverse priming site directed to the other end of that amplicon. Thereafter, the experimentalist can dilute the reaction mixture to single molecules and perform a universal PCR, such as a universal emulsion PCR, wherein the emulsion comprises a universal forward primer and a universal reverse primer. Examples of emulsion PCR can be found, for example, in Dressman et al., WO2005/010145, Published US Patent Application US20070065823A1, and PNAS 100:15:8817-8822 (2003). Thereafter, sequencing reactions can be performed.

In some embodiments, one can perform a PCR using target sequence-specific primers that can lack a tail. The resulting amplicon, bearing template independent adenine at its 3′ ends, can be used in “T/A cloning” approach. For example, the template independent A's can be used as sticky ends to ligate adapters.

The adapters comprise cleavage-related moieties such as type IIs restriction sites, as well as sticky ends bearing a T. Following ligation of these adapters, a digestion with a cleavage agent such as a type IIs restriction enzyme can be performed to remove the primer specific portion of the amplicon. Thereafter, an additional ligation reaction can be performed wherein adapters comprising sequencing primer portions are ligated to the ends of the digested amplicons.

In some embodiments, the present teachings provide use of “TOPO” cloning, by using a vector that has one or two Type IIs sites flanking the cloning site. Such approaches can allow for the placing Type IIs sites next to the primer sequences, but in a manner that does not employ ligation. Topo cloning kits are commercially available from, for example, Invitrogen. Illustrative teachings of such approaches can be found, for example, in Shuman, S. (1994) J. Biol. Chem. 269: 32678-32684 and Bernard, P. et al. (1994) Gene 148: 71-74. In some embodiments, directional versions of TOPO approaches can be employed, as are commercially available from Invitrogen, where it is referred to as Directional (Topo Cloning.

In some embodiments, the cleavage of an amplicon results in removal of all of the target-specific portion of a primer employed in an original amplification reaction. In some embodiments, the cleavage of the amplicon result results in removal of all but one base of the target-specific portion of the primer employed in the original amplification reaction. In some embodiments, the cleavage of the amplicon result results in removal of all but two bases of the target-specific portion of the primer employed in the original amplification reaction. In some embodiments, the cleavage of the amplicon result results in removal of all but three bases of the target-specific portion of the primer employed in the original amplification reaction. In some embodiments, the cleavage of the amplicon result results in removal of all but four bases of the target-specific portion of the primer employed in the original amplification reaction. In some embodiments, the cleavage of the amplicon result results in removal of all but five bases of the target-specific portion of the primer employed in the original amplification reaction. In some embodiments, the cleavage of the amplicon result results in removal of all but six bases of the target-specific portion of the primer employed in the original amplification reaction. In some embodiments, the cleavage of the amplicon result results in removal of all but seven bases of the target-specific portion of the primer employed in the original amplification reaction. In some embodiments, the cleavage of the amplicon result results in removal of all but eight bases of the target-specific portion of the primer employed in the original amplification reaction. In some embodiments, the cleavage of the amplicon result results in removal of all but nine bases of the target-specific portion of the primer employed in the original amplification reaction. In some embodiments, the cleavage of the amplicon result results in removal of all but ten bases of the target-specific portion of the primer employed in the original amplification reaction.

In some embodiments, the present teachings can be employed to amplify a messenger RNA (mRNA) comprising a poly-A tail. For example, a reverse transcription primer bearing an oligo-dt region that is complementary to the poly-A tail of the mRNA can be employed, where such a reverse transcription primer further contains a cleavage-related moiety. Such a primer can further comprise at least one non-dt nucleotide at its 3′end, so that the primer hybridizes at the interface of the poly-A tail and the non-poly-A tail of the mRNA. Thus, cleavage of a resulting amplicon, for example with a type IIs restriction enzyme, will liberate sequence information within the mRNA that is not poly-A tail. Approaches for designing primers for reverse transcribing mRNA bearing poly-A tails in a manner to query the interface of the poly-A tail and the non-poly-A tail of the mRNA are routinely used, for example in differential display PCR as described in U.S. Pat. No. 5,262,311, U.S. Pat. No. 5,559,672, and U.S. Pat. No. 5,665,547.

### Example

A 34 bp region (34 bp excluding primer sequences) of BRCA1 is amplified using a forward primer (SEQ ID NO:1) and a reverse primer (SEQ ID NO:2).

| | |
|---|---|
| SEQ ID NO: 1 | 5′GCCTCCAACggaaaccagtctcagtgtcca |
| SEQ ID NO: 2 | 5′CCGTCCGAGggttgtatccgctgctttgt |

As shown, the lower case bases indicate the target specific portion of the primer that is specific for the BRCA1 sequence. The underlined sequence is the MmeI recognition site, and the extra 3 bases at the 5′ end can be added to improve the efficiency of MmeI digestion by increasing the ds DNA footprint, and increasing the stability of the duplex near the end.

The PCR reaction contains:

25 ul 2× AmpliTaq Gold master mix

13 ul water

2 ul 5 ng/ul human genomic DNA

5 ul 2 uM forward primer

5 ul 2 uM reverse primer

The reaction is incubated at 95° C. for 10 minutes, then cycled 30 times: 95° C. for 15 seconds, 60° C. for 1 minute.

Following the PCR, the amplicon is purified using a Qiagen MinElute PCR purification spin column according to the manufacturer's instructions, resulting in ˜9 ul purified amplicon.

The amplicon is digested with MmeI (New England Biolabs) as follows:

9 ul purified amplicon

6 ul water

2 ul 10× NEBuffer 2

2 uL 500 uM S-adenosylmethionine

1 uL 2 units/ul MmeI

The reaction is incubated at 37° C. for 15 minutes, then purified with a Qiagen MinElute PCR purification spin column according to the manufacturer's instructions, resulting in ˜9 ul purified digested amplicon.

The digested amplicon now has two base 3′ overhangs of GG from the forward primer and AC from the reverse primer. These are ligated to double stranded adaptors containing sequencing primer sites as follows:

| | |
|---|---|
| 9 ul | purified digested amplicon |
| 5 ul | water |
| 20 ul | 2X Quick Ligation Kit buffer (New England |
| | Biolabs) |
| 1 ul | 8 uM M13-F adaptor |

| | |
|---|---|
| SEQ ID NO: 3 | 5′TGTAAAACGACGGCCAGTCC) |

| | |
|---|---|
| 1 ul | 8 uM cM13-F adaptor |

| | |
|---|---|
| SEQ ID NO: 4 | 5′-PhosACTGGCCGTCGTTTTACA |

| | |
|---|---|
| 1 ul | 8 uM M13-R adaptor |

| | |
|---|---|
| SEQ ID NO: 5 | 5′-CAGGAAACAGCTATGACCGT |

| | |
|---|---|
| 1 ul | 8 uM cM13-R adaptor |

| | |
|---|---|
| SEQ ID NO: 6 | 5′-PhosGGTCATAGCTGTTTCCTG |

| | |
|---|---|
| 2 ul | Quick T4 DNA Ligase |

The reaction is incubated at 25° C. for 15 minutes, then purified with a Qiagen MinElute PCR purification spin column according to the manufacturer's instructions, resulting in ˜9 ul purified sequencing primer adapted-amplicon. This amplicon is now ready for sequencing using M13-forward (SEQ ID NO:7) or M13-reverse (SEQ ID NO: 8) sequencing primers.

| | |
|---|---|
| SEQ ID NO: 7 | 5′TGTAAAACGACGGCCAGT |
| SEQ ID NO: 8 | 5′CAGGAAACAGCTATGACC |

Additional teachings regarding various molecular biology approaches, including PCR and nucleic acid sequencing, as well as general definitions applicable herein, can be found in Sambrook and Russell, Molecular Cloning, A Laboratory Manual, 3rd Edition, as well as on the internet at www.molecularcloning.com.

Exemplary Kits in Accordance with Some Embodiments of the Present Teachings

In some embodiments, the present teachings also provide kits designed to expedite performing certain methods. In some embodiments, kits serve to expedite the performance of the methods of interest by assembling two or more components used in carrying out the methods. In some embodiments, kits may contain components in pre-measured unit amounts to minimize the need for measurements by end-users. In some embodiments, kits may include instructions for performing one or more methods of the present teachings. In certain embodiments, the kit components are optimized to operate in conjunction with one another.

In some embodiments, the present teachings provide a kit comprising; a forward primer comprising a target-specific portion and a 5′ tail, wherein the 5′ tail of the forward primer comprises a type IIs restriction enzyme recognition site; a reverse primer comprising a target-specific portion and a 5′ tail, wherein the 5′ tail of the reverse primer comprises a type IIs restriction enzyme recognition site; a first adapter comprising a first universal primer portion; a second adapter comprising a second universal primer portion; a ligase; a polymerase; and, dNTPs. In some embodiments, such a kit further comprises nucleotide terminators. In some embodiments, such a kit further comprises a first sequencing primer and a second sequencing primer, wherein the first sequencing primer is encoded by the first universal primer portion of the first adapter and wherein the second sequencing primer is encoded by the second universal primer portion of the second adapter. In some embodiments, a kit can comprise the first sequencing primer, the second sequencing primer, or both the first sequencing primer and the second sequencing primer containing a fluorescent label. In some embodiments, such a kit further comprises sequencing ligation probes.

In some embodiments, the present teachings provide a kit comprising; a forward primer comprising a target-specific portion and a 5′ tail, wherein the 5′ tail of the forward primer comprises an exonuclease-resistant moiety; a reverse primer comprising a target-specific portion and a 5′ tail, wherein the 5′ tail of the reverse primer comprises a exonuclease-resistant moiety; a first adapter comprising a first universal primer portion; a second adapter comprising a second universal primer portion; a ligase; a polymerase; and, dNTPs. Some embodiments of such kits comprise nucleotide terminators. Some embodiments of such kits comprise a first sequencing primer and a second sequencing primer, wherein the first sequencing primer is encoded by the first universal primer portion of the first adapter and wherein the second sequencing primer is encoded by the second universal primer portion of the second adapter. In some embodiments of such kits, the first sequencing primer, the second sequencing primer, or both the first sequencing primer and the second sequencing primer contain a fluorescent label. Some embodiments of such kits comprise sequencing ligation probes.

In some embodiments, the present teachings provide a kit comprising; a forward primer comprising a target-specific portion and a 5′ tail, wherein the 5′ tail of the forward primer comprises cleavage-related moiety; a reverse primer comprising a target-specific portion and a 5′ tail, wherein the 5′ tail of the reverse primer comprises a cleavage-related moiety; a first adapter comprising a first universal primer portion; a second adapter comprising a second universal primer portion; a ligase; a polymerase; and, dNTPs. In some embodiments, such kits comprise nucleotide terminators. In some embodiments, such kits comprise a first sequencing primer and a second sequencing primer, wherein the first sequencing primer is encoded by the first universal primer portion of the first adapter and wherein the second sequencing primer is encoded by the second universal primer portion of the second adapter. In some embodiments, kits can comprise the first sequencing primer, the second sequencing primer, or both the first sequencing primer and the second sequencing primer containing a fluorescent label. In some embodiments, such kits comprise a cleavage-related moiety that is a type IIs restriction enzyme recognition site. In some embodiments, such kits comprise a cleavage-related moiety that is an exonuclease resistant moiety. In some embodiments, such kits comprise sequencing ligation probes.

The present teachings also provide novel compositions. For example, in some embodiments, the present teachings provide a composition comprising; a PCR amplicon less than 50 nucleotides in length, the PCR amplicon comprising a first single-stranded end resulting from cleavage of a first type IIs restriction enzyme recognition site by a type IIs restriction enzyme that was introduced by a forward primer, and, a second single-stranded end resulting from cleavage of a type IIs restriction enzyme site by a type IIs restriction enzyme that was introduced by a reverse primer. In some composition embodiments, the first single-stranded end is 1-4 bases in length, the second single-stranded end is 1-4 bases in length, or the first single-stranded end is 1-4 bases in length and the second single-stranded end are both 1-4 bases in length. In some composition embodiments, the first single-stranded end, the second single-stranded end, or both the first single-stranded end and the second single-stranded end result from cleavage with Mme I or EcoP15I.

In some embodiments, the present teachings provide a composition comprising; a PCR amplicon less than 50 nucleotides in length, the PCR amplicon comprising a first single-stranded end resulting from cleavage with a first exonuclease of a region upstream from a exonuclease-resistant moiety that was introduced by a forward primer, and, a second single-stranded end resulting from cleavage with a second exonuclease of a region upstream from a exonuclease-resistant moiety that was introduced by a reverse primer. In some composition embodiments, the first single-stranded end is 1-4 bases in length, the second single-stranded end is 1-4 bases in length, or the first single-stranded end is 1-4 bases in length and the second single-stranded end are both 1-4 bases in length. In some composition embodiments, the first exonuclease and the second exonuclease are the same exonuclease.

In another composition embodiment, the present teachings provide a composition comprising; a digested amplicon ligated to an adapter, and, a sequencing primer hybridized to the adapter, wherein the digested amplicon is less than 50 nucleotides in length and comprises a single-stranded end hybridized to the adapter, wherein the single-stranded end results from cleavage of a cleavage-related moiety introduced by an amplification primer. In some embodiments, the cleavage-related moiety introduced by the amplification primer is a type IIs restriction enzyme recognition site. In some embodiments, the cleavage-related moiety introduced by the amplification primer is an exonuclease resistant moiety. In some embodiments, the amplification primer is a PCR primer, and wherein the digested amplicon is a digested PCR amplicon. In some embodiments, the amplification primer is a rolling circle amplification primer, and wherein the digested amplicon is a digested rolling circle amplification reaction product.

While the present teachings have been described in terms of these exemplary embodiments, the skilled artisan will readily understand that numerous variations and modifications of these exemplary embodiments are possible without undue experimentation. All such variations and modifications are within the scope of the present teachings. Aspects of the present teachings may be further understood in light of the following claims.

We claim:

1. A method of performing a re-sequencing reaction comprising;
performing a polymerase chain reaction (PCR) with a forward primer and a reverse primer to form a PCR amplicon,
wherein the forward primer comprises a target-specific portion and a 5′ tail, wherein the 5′ tail of the forward primer comprises a type IIs restriction enzyme recognition site, and,
wherein the reverse primer comprises a target-specific portion and a 5′ tail, wherein the 5′ tail of the reverse primer comprises a type IIs restriction enzyme recognition site;
digesting the PCR amplicon with a type IIs restriction enzyme to form a digested amplicon with a first end and a second end;
ligating an adapter to the first end of the digested amplicon and ligating an adapter to the second end of the digested amplicon to form an adapter-ligated amplicon with a first end and a second end; and,
sequencing the adapter-ligated amplicon.

2. The method according to claim 1 wherein the sequencing comprises
hybridizing a primer to the first end of the adapter-ligated amplicon; and,
performing a chain termination reaction.

3. The method according to claim 1 wherein the sequencing comprises a cycled ligation.

4. The method according to claim 1 wherein the adapter ligated to the first end of the digested amplicon is different from the adapter ligated to the second end of the digested amplicon.

5. The method according to claim 1 wherein the type IIs restriction enzyme is Mme I or EcoP15I.

6. A method of forming an adapter-ligated amplicon comprising a first end that differs in sequence from a second end, said method comprising;
amplifying a short nucleic acid in a polymerase chain reaction (PCR) comprising a forward primer and a reverse primer to form a PCR amplicon,
wherein the forward primer comprises a target-specific portion and a 5′ tail, wherein the 5′ tail of the forward primer comprises a type IIs restriction enzyme recognition site, and,
wherein the reverse primer comprises a target-specific portion and a 5′ tail, wherein the 5′ tail of the reverse primer comprises a type IIs restriction enzyme recognition site;
digesting the PCR amplicon with a type IIs restriction enzyme to form a digested amplicon with a first end and a second end; and,
ligating a first adapter to the first end of the digested amplicon and ligating a second adapter to the second end of the digested amplicon to form an adapter-ligated amplicon with a first end that differs in sequence from a second end.

7. The method according to claim 6 wherein the PCR amplicon is 18-100 nucleotides in length.

8. The method according to claim 6 wherein the type IIs restriction enzyme is Mme I or EcoP15I.

9. A method of increasing the percent of novel sequence information in a sequencing reaction of a digested PCR amplicon less than 100 bases in length comprising;
performing a polymerase chain reaction (PCR) with a forward primer and a reverse primer to form a PCR amplicon,
wherein the forward primer comprises a target-specific portion and a 5′ tail, wherein the 5′ tail of the forward primer comprises a type IIs restriction enzyme recognition site, and,
wherein the reverse primer comprises a target-specific portion and a 5′ tail, wherein the 5′ tail of the reverse primer comprises a type IIs restriction enzyme recognition site;
digesting the PCR amplicon with a type IIs restriction enzyme to form a digested PCR amplicon with a first end and a second end, wherein the digested PCR amplicon is less than one hundred bases in length;
ligating an adapter to the first end of the digested PCR amplicon and ligating an adapter to the second end of the digested PCR amplicon to form an adapter-ligated PCR amplicon with a first end and a second end;
sequencing the adapter-ligated amplicon; and,
increasing the percent of novel sequence information in a sequencing reaction of the PCR amplicon as compared to sequencing a PCR amplicon that was amplified in a PCR not comprising primers with a type IIs restriction enzyme site.

10. The method according to claim 9 wherein the sequencing comprises
hybridizing a primer to the first end of the adapter-ligated amplicon; and,
performing a chain termination reaction.

11. The method according to claim 9 wherein the sequencing comprises a cycled ligation.

12. The method according to claim 9 wherein the adapter ligated to the first end of the digested PCR amplicon is different from the adapter ligated to the second end of the digested PCR amplicon.

13. The method according to claim 1 wherein the type IIs restriction enzyme is Mme I or EcoP151.

14. A method of sequencing a target polynucleotide comprising;
amplifying the target polynucleotide with at least one primer to form an amplicon comprising a type IIs restriction enzyme recognition site on at least one end;
digesting the PCR amplicon with a type IIs restriction enzyme to form a digested amplicon with at least one ligatable end;
ligating an adapter to the ligatable end of the digested amplicon to form an adapter-ligated amplicon with a known end;
hybridizing a sequencing primer to the known end; and,
sequencing the adapter-ligated amplicon, wherein the first sequenced base is target polynucleotide information.

15. The method according to claim 14 wherein the sequencing comprises
hybridizing a primer to the first end of the adapter-ligated amplicon; and,
performing a chain termination reaction.

16. The method according to claim 14 wherein the sequencing comprises a cycled ligation.

17. The method according to claim 14 wherein the type IIs restriction enzyme is Mme I or EcoP151.

18. A method of performing a sequencing reaction comprising;
performing a polymerase chain reaction (PCR) with a forward primer and a reverse primer to form a PCR amplicon,
wherein the forward primer comprises a target-specific portion and a 5′ tail, wherein the 5′ tail of the forward primer comprises an exonuclease-resistant moiety, and,
wherein the reverse primer comprises a target-specific portion and a 5′ tail, wherein the 5′ tail of the reverse primer comprises an exonuclease-resistant moiety;
digesting the PCR amplicon with an exonuclease to form a digested PCR amplicon with a first end and a second end;
ligating an adapter to the first end of the digested PCR amplicon and to the second end of the digested PCR amplicon to form an adapter-ligated amplicon with a first end and a second end; and,
sequencing the adapter-ligated amplicon.

19. The method according to claim 18 wherein the sequencing comprises
hybridizing a primer to the first end of the adapter-ligated amplicon; and,
performing a chain termination reaction.

20. The method according to claim 18 wherein the sequencing comprises a cycled ligation.

21. The method according to claim 18 wherein the adapter ligated to the first end of the digested amplicon comprises a sequence that is different from the adapter ligated to the second end of the digested amplicon.

22. The method according to claim 1 wherein the exonuclease is lambda exonuclease.

23. A method of performing a sequencing reaction, wherein the first sequenced base is target polynucleotide information, said method comprising;
performing a primer-mediated amplification reaction to form an amplicon, wherein the primer comprises a target-specific portion and cleavage-related moiety;
digesting the amplicon with a cleavage agent directed to the cleavage-related moiety to form a digested amplicon with a cleaved end;
ligating an adapter to the cleaved end of the digested amplicon to form an adapter-ligated amplicon; and,
sequencing the adapter-ligated amplicon, wherein the first sequenced base is target polynucleotide information.

24. The method according to claim 23 wherein the sequencing comprises
hybridizing a primer to the first end of the adapter-ligated amplicon; and,
performing a chain termination reaction.

25. The method according to claim 23 wherein the sequencing comprises a cycled ligation.

26. The method according to claim 23 wherein the cleavage-related moiety is a type IIs restriction enzyme recognition site.

27. The method according to claim 26 wherein the type IIs restriction enzyme is Mme I or EcoP15I.

28. The method according to claim 23 wherein the cleavage-related moiety is an exonuclease-resistant moiety.

29. The method according to claim 28 wherein the exonuclease-resistant moiety is polyethylene glycol (PEG).

30. A kit comprising;
a forward primer comprising a target-specific portion and a 5′ tail, wherein the 5′ tail of the forward primer comprises a type IIs restriction enzyme recognition site;
a reverse primer comprising a target-specific portion and a 5′ tail, wherein the 5′ tail of the reverse primer comprises a type IIs restriction enzyme recognition site;
a first adapter comprising a first universal primer portion;
a second adapter comprising a second universal primer portion;
a ligase;
a polymerase; and,
dNTPs.

31. The kit according to claim 30 further comprising nucleotide terminators.

32. The kit according to claim 30 further comprising a first sequencing primer and a second sequencing primer, wherein the first sequencing primer is encoded by the first universal primer portion of the first adapter and wherein the second sequencing primer is encoded by the second universal primer portion of the second adapter.

33. The kit according to claim 32 wherein the first sequencing primer, the second sequencing primer, or both the first sequencing primer and the second sequencing primer contain a fluorescent label.

34. The kit according to claim 30 further comprising sequencing ligation probes.

35. A composition comprising;
a digested PCR amplicon less than 50 nucleotides in length, the digested PCR amplicon comprising a first single-stranded end resulting from cleavage of a first type IIs restriction enzyme recognition site by a type IIs restriction enzyme that was introduced by a forward primer, and, a second single-stranded end resulting from cleavage of a type IIs restriction enzyme site by a type IIs restriction enzyme that was introduced by a reverse primer.

36. The composition according to claim 35 where the first single-stranded end is 1-4 bases in length, the second single-stranded end is 1-4 bases in length, or the first single-stranded end is 1-4 bases in length and the second single-stranded end are both 1-4 bases in length.

37. The composition according to claim 35 wherein the first single-stranded end, the second single-stranded end, or both the first single-stranded end and the second single-stranded end result from cleavage with Mme I or EcoP15I.

38. A kit comprising;
a forward primer comprising a target-specific portion and a 5′ tail, wherein the 5′ tail of the forward primer comprises a exonuclease-resistant moiety;
a reverse primer comprising a target-specific portion and a 5′ tail, wherein the 5′ tail of the reverse primer comprises an exonuclease-resistant moiety;
a first adapter comprising a first universal primer portion;
a second adapter comprising a second universal primer portion;
a ligase;
a polymerase; and,
dNTPs.

39. The kit according to claim 38 further comprising nucleotide terminators.

40. The kit according to claim 38 further comprising a first sequencing primer and a second sequencing primer, wherein the first sequencing primer is encoded by the first universal primer portion of the first adapter and wherein the second sequencing primer is encoded by the second universal primer portion of the second adapter.

41. The kit according to claim 40 wherein the first sequencing primer, the second sequencing primer, or both the first sequencing primer and the second sequencing primer contain a fluorescent label.

42. The kit according to claim 38 further comprising sequencing ligation probes.

43. A composition comprising;
a digested PCR amplicon less than 50 nucleotides in length, the digested PCR amplicon comprising a first single-stranded end resulting from cleavage with a first exonuclease of a region upstream from a first exonuclease-resistant moiety that was introduced by a forward primer, and, a second single-stranded end resulting from cleavage with a second exonuclease of a region upstream from a second exonuclease-resistant moiety that was introduced by a reverse primer.

44. The composition according to claim 43 where the first single-stranded end is 1-4 bases in length, the second single-stranded end is 1-4 bases in length, or the first single-stranded end is 1-4 bases in length and the second single-stranded end are both 1-4 bases in length.

45. The composition according to claim 43 wherein the first exonuclease and the second exonuclease are the same exonuclease.

46. A kit comprising;
a forward primer comprising a target-specific portion and a 5′ tail, wherein the 5′ tail of the forward primer comprises cleavage-related moiety;
a reverse primer comprising a target-specific portion and a 5′ tail, wherein the 5′ tail of the reverse primer comprises a cleavage-related moiety;
a first adapter comprising a first universal primer portion;
a second adapter comprising a second universal primer portion;
a ligase;
a polymerase; and,
dNTPs.

47. The kit according to claim 46 further comprising nucleotide terminators.

48. The kit according to claim 46 further comprising a first sequencing primer and a second sequencing primer, wherein the first sequencing primer is encoded by the first universal primer portion of the first adapter and wherein the second sequencing primer is encoded by the second universal primer portion of the second adapter.

49. The kit according to claim 48 wherein the first sequencing primer, the second sequencing primer, or both the first sequencing primer and the second sequencing primer contain a fluorescent label.

50. The kit according to claim 46 wherein the cleavage-related moiety is a type IIs restriction enzyme recognition site.

51. The kit according to claim 46 wherein the cleavage-related moiety is an exonuclease resistant moiety.

52. The kit according to claim 46 further comprising sequencing ligation probes.

53. A composition comprising;
a digested amplicon ligated to an adapter, and,
a sequencing primer hybridized to the adapter,
wherein the digested amplicon is less than 50 nucleotides in length and comprises a single-stranded end hybridized to the adapter, wherein the single-stranded end results from cleavage of a cleavage-related moiety introduced by an amplification primer.

54. The composition according to claim 53 wherein the cleavage-related moiety introduced by the amplification primer is a type IIs restriction enzyme recognition site.

55. The composition according to claim 53 wherein the cleavage-related moiety introduced by the amplification primer is an exonuclease resistant moiety.

56. The composition according to claim 53 wherein the amplification primer is a PCR primer, and wherein the digested amplicon is a digested PCR amplicon.

57. The composition according to claim 53 wherein the amplification primer is a rolling circle amplification primer, and wherein the digested amplicon is a digested rolling circle amplification reaction product.
